(19) United States
(12) Patent Application Publication
(10) Pub. No.: US 2014/0225534 A1
(43) Pub. Date: Aug. 14, 2014

(54) MULTI-JOINT RADIOTHERAPY APPARATUS USING FLEXIBLE AND ROTARY COUPLING WAVEGUIDE PIPE

(71) Applicant: Korea Atomic Energy Research Institute, Daejeon (KR)

(72) Inventors: Byung Cheol Lee, Daejeon (KR); Byeong-No Lee, Daejeon (KR); Seungho Kim, Daejeon (KR); Kyung-min Jeong, Daejeon (KR); JongSeo Chai, Suwon-si (KR); Ki Baek Song, Daejeon (KR); Hyung Dal Park, Daejeon (KR); SeungWook Shin, Suwon-si (KR); SeungHyun Lee, Suwon-si (KR); JongChul LEE, Suwon-si (KR)

(73) Assignee: Korea Atomic Energy Research Institute, Daejeon (KR)

(21) Appl. No.: 14/179,463

(22) Filed: Feb. 12, 2014

(30) Foreign Application Priority Data
Feb. 13, 2013 (KR) 10-2013-0015420

(51) Int. Cl. A61N 5/10

(52) U.S. Cl. CPC A61N 5/1083
USPC 315/505

(57) ABSTRACT

A radiotherapy apparatus includes a beam irradiation head (10), a linear accelerating means (20) provided at the beam irradiation head (10), a robot arm (30) connected with the beam irradiation head (10) and having a plurality of joints, a waveguide (40) built in the robot arm 30 and connected with the linear accelerating means (20), and an electromagnetic wave oscillator (50) disposed outside the robot arm (30) and generating electromagnetic waves so that the electromagnetic waves are propagated to the linear accelerating means (20) through the waveguide (40).

### CROSS-REFERENCE TO RELATED APPLICATION

This application claims priority to and the benefit of Korean Patent Application No. 10-2013-0015420, filed on Feb. 13, 2013, the disclosure of which is incorporated herein by reference in its entirety.

### BACKGROUND

1. Field

The present disclosure relates to a multi-joint radiotherapy apparatus using a flexible and rotary-coupling waveguide pipe.

2. Discussion of Related Art

A conventional radiotherapy apparatus is disclosed in U.S. Pat. No. 7,759,883 (Jul. 20, 2010, hereinafter, called “conventional technique”) entitled “DUAL-ROTARY-COUPLING INTERNAL-WAVEGUIDE LINAC FOR IOTR”.

The conventional technique relates to a multi-joint radiotherapy apparatus. A connection part of a waveguide is formed into a rotary-coupling type in order to install the waveguide in a robot arm.

However, in the conventional technique, since an electromagnetic wave oscillator is located in the robot arm, a weight of the robot arm is increased, and thus it is not easy to achieve an inertia control of an irradiation head.

Further, when the weight of the robot arm is increased, it is not easy to precisely control the robot arm, and it is difficult to irradiate radiation to an exact position of an affected part, and thus it is difficult to irradiate a consistent amount of radiation.

### SUMMARY

Therefore, it is an aspect of the present invention to provide a multi-joint radiotherapy apparatus using a flexible and rotary-coupling waveguide pipe, in which an electromagnetic wave oscillator and a beam irradiation head are separated from each other, and the waveguide propagating electromagnetic waves generated from the electromagnetic wave oscillator to a linear accelerating means is built in a robot arm supporting the beam irradiation head, such that a weight of the beam irradiation head and a load applied to a multi-joint robot arm may be reduced, and also which enables stereotactic radiotherapy having the same center through an accurate operation control and thus always obtains a consistent dose, even though a beam is irradiated at any positions.

It is another aspect of the present invention to provide a multi-joint radiotherapy apparatus using a flexible and rotary-coupling waveguide pipe, in which a rotary-coupling waveguide pipe is provided to a rotary-coupling part of each joint of a robot arm so that a motion at each joint is smoothly performed, and the waveguide has a straight pipe structure or a flexible pipe structure, and thus it is possible to provide the waveguide fitting motion characteristics.

It is yet another aspect of the present invention to provide a multi-joint radiotherapy apparatus using a flexible and rotary-coupling waveguide pipe, in which a wave passage of the waveguide has a rectangular shape so as to reduce a propagation loss of radiofrequency waves.

According to an aspect of the present invention, there is provided a multi-joint radiotherapy apparatus using a flexible and rotary-coupling waveguide pipe, including a beam irradiation head (10), a linear accelerating means (20) provided at the beam irradiation head (10), a robot arm (30) connected with the beam irradiation head (10) and having a plurality of joints, a waveguide (40) built in the robot arm (30) and connected with the linear accelerating means (20), and an electromagnetic wave oscillator (50) disposed outside the robot arm (30) and generating electromagnetic waves so that the electromagnetic waves are propagated to the linear accelerating means (20) through the waveguide (40).

### BRIEF DESCRIPTION OF THE DRAWINGS

The above and other features and advantages of the present invention will become more apparent to those of ordinary skill in the art by describing in detail exemplary embodiments thereof with reference to the accompanying drawings, in which:

FIG. 1 is a perspective view of a multi-joint radiotherapy apparatus using a flexible and rotary-coupling waveguide pipe according to embodiments of the present invention;

FIGS. 2A and 2B are conceptual views of various waveguides of the multi-joint radiotherapy apparatus according to embodiments of the present invention; and

FIGS. 3A, 3B and 3C are conceptual views of various rotary-coupling waveguide pipes of the multi-joint radiotherapy apparatus according to embodiments of the present invention.

### DETAILED DESCRIPTION OF EXEMPLARY EMBODIMENTS

Exemplary embodiments of the present invention will be described in detail below with reference to the accompanying drawings. While the present invention is shown and described in connection with exemplary embodiments thereof, it will be apparent to those skilled in the art that various modifications can be made without departing from the spirit and scope of the invention.

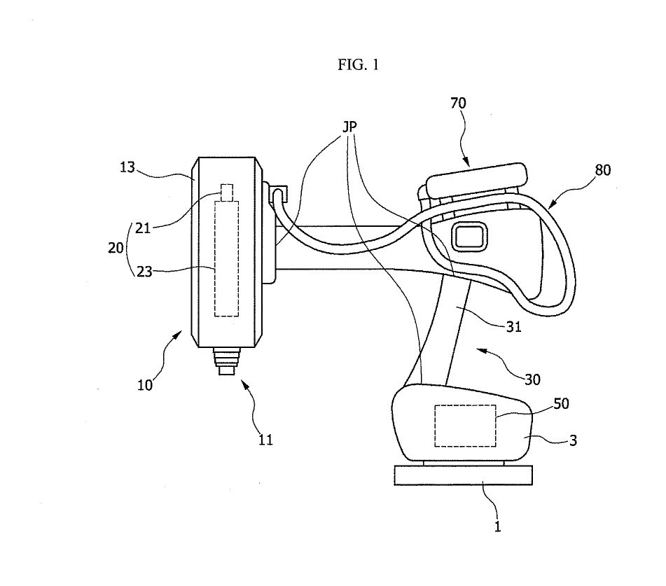

As illustrated in FIGS. 1 to 3, a radiotherapy apparatus according to embodiments of the present invention includes a beam irradiation head 10, a linear accelerating means 20, a multi-joint robot arm 30, an electromagnetic wave oscillator 50, and a waveguide 40 which propagates electromagnetic waves from the electromagnetic wave oscillator 50 to the linear accelerating means 20.

The beam irradiation head 10, as illustrated in FIG. 1, includes a housing 13 which is connected to the robot arm 30 to be supported, and a collimator 11 which is disposed at a lower end of the housing 13 to restrict an irradiation direction of radiation and a diffusion thereof. Also, the beam irradiation head 10 is configured so that an irradiation dose of other light except a used beam is released as small as possible.

To this end, the collimator 11 is made of a radiation absorbent material such as lead and tungsten, and, if necessary, may have a different thickness and size.

A conventional beam irradiation head includes the linear accelerating means 20 which accelerates and supplies electrons so that radiation is irradiated from the collimator 11, and the electromagnetic wave oscillator 50 which generates electromagnetic waves and supplies the electromagnetic waves to the electrons accelerated by the linear accelerating means 20.

Here, the linear accelerating means 20 includes an electron gun 21 which generates electrons, and an accelerating pipe 23 which accelerates the electrons generated from the electron gun 21. The linear accelerating means 20 is built in the housing 13.

And the electromagnetic wave oscillator 50 includes an RF generator generating electromagnetic waves, i.e., radiofrequency waves, and the RF generator is built in the housing 13 so that the accelerating pipe 23 is connected with the waveguide 40 propagating the electromagnetic waves.

That is, as described above, the conventional beam irradiation head includes the linear accelerating means 20, the electromagnetic wave oscillator 50, the waveguide 40, and so on.

Particularly, in case of the electromagnetic wave oscillator 50, the linear accelerating means 20 has a small size, but reaches a weight of about 30 kg.

Therefore, since a total weight of the linear accelerating means 20, the electromagnetic wave oscillator 50, and the waveguide 40 is applied to the robot arm 30 supporting the beam irradiation head 10, a supporting load of the robot arm 30 is increased.

This makes an inertia control difficult and thus makes an accurate control difficult, when the robot arm 30 is operated. Therefore, it is impossible to perform stereotactic radiotherapy having the same center, and also it is difficult to obtain a consistent dose according to an irradiation position of the beam irradiation head 10.

To overcome the above-mentioned disadvantages, in embodiments of the present invention, the electromagnetic wave oscillator 50 and the beam irradiation head 10 are configured to be separated from each other and thus reduce a weight of the beam irradiation head 10. Therefore, since the supporting load of the robot arm 30 is reduced, it is possible to perform the stereotactic radiotherapy having the same center, and also obtain a consistent dose according to the irradiation position of the beam irradiation head 10.

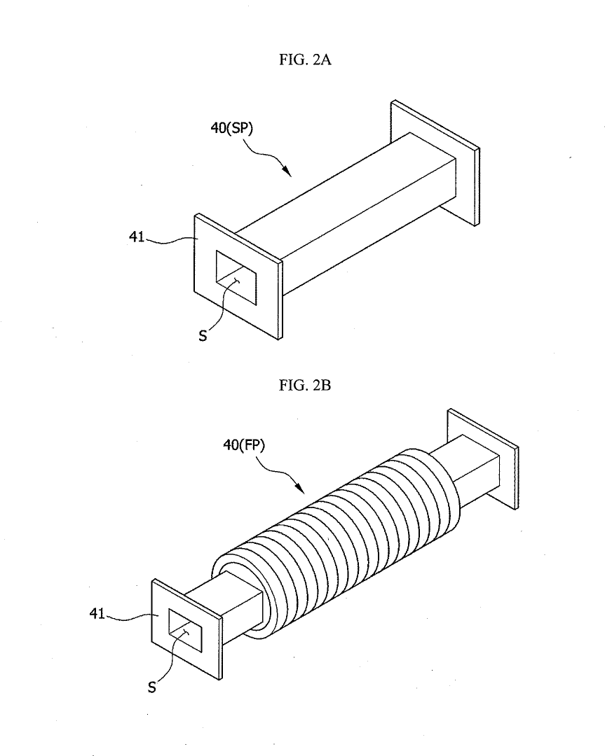

Then, as illustrated in FIGS. 1 and 2, when the electromagnetic wave oscillator 50 is separated from the beam irradiation head 10, the waveguide 40 is provided to propagate the electromagnetic waves, i.e., the radiofrequency waves generated from the electromagnetic wave oscillator 50 to the linear accelerating means 20. At this time, the waveguide 40 (or a waveguide pipe) is built in the robot arm 30 having multiple joints.

That is, the robot arm 30 is configured such that multiple arms 31 are connected to each other through the multiple joints. Therefore, it is possible to perform a joint movement such as a rotary motion and thus precisely irradiate a beam from the collimator 11 of the beam irradiation head 10 to an affected part.

Further, the waveguide 40 has a wave passage S through which the electromagnetic waves are moved. At this time, the waveguide 40 has a straight pipe structure SP (FIG. 2A) or a flexible pipe structure FP (FIG. 2B) and is built in each arm 31 of the robot arm 30.

In this case, since the flexible pipe FP type waveguide 40 has flexibility, the waveguide 40 may be applied in various forms according to installation characteristics, such as a case that it is necessary to precisely adjust an alignment line of the waveguide 40 and a case of requiring a bending motion.

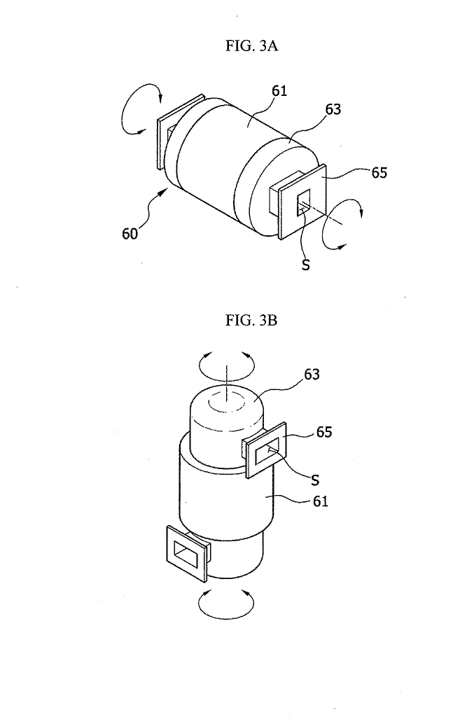

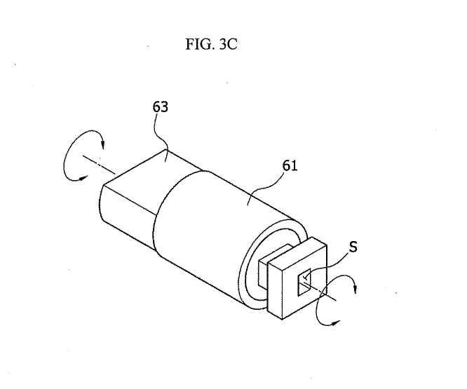

And as illustrated in FIG. 3, a rotary-coupling waveguide pipe 60 is provided at each joint part JP of the robot arm 30 so as to embody a smooth rotary motion of the robot arm 30.

According to a position of the joint part JP of the robot arm 30, the rotary-coupling waveguide pipe 60 may be connected with an adjacent end of the waveguide 40 built in each arm 31, may be connected with the accelerating pipe 23 at an upper end of the robot arm 30, or may be connected with the electromagnetic wave oscillator 50 at a lower end of the robot arm 30, such that the rotary motion may be smoothly performed at each joint part JP.

Also, the rotary-coupling waveguide pipe 60 has a wave passage S so that propagation of the electromagnetic waves may be maintained.

That is, the rotary-coupling waveguide pipe 60 includes a body 61 which is disposed at each joint part JP, and a rotary-coupling part 63 which is rotatably disposed at one end or both ends of the body 61.

Further, the body 61 and the rotary-coupling part 63 have the wave passage S communicating with the wave passage S of the waveguide 40 and forming a passage of the electromagnetic waves.

Particularly, the rotary-coupling part 63 may be formed into a rotary joint and may be provided to both ends of the body 61. Since the rotary motion is performed at each joint part JP of the robot arm 30, i.e., a connection part between the beam irradiation head 10 and the robot arm 30, a connection part of each arm 31, and a connection part between the electromagnetic wave oscillator 50 and the robot arm 30, the rotary-coupling part 63 may be provided to the both ends of each body 61.

The rotary-coupling part 63 may be prepared into various forms, such as a straight connection form (FIG. 3A), a U-shaped connection form (FIG. 3B), and an L-shaped connection form (FIG. 3C), according to connection forms and motion characteristics of objects to be connected.

Further, a counter-flange 41 coupled to a flange provided at the rotary-coupling part 63 is provided at both ends of the waveguide 40 and fastened through a bolt and a nut.

In this case, when the rotary-coupling waveguide pipe 60 is fixedly built in the joint part and the waveguide is inserted into each arm, each flange may be coupled thereto.

In addition, the wave passage S of the waveguide 40 and the rotary-coupling waveguide pipe 60 may have a rectangular shape. This is because each surface of the wave passage S may be formed to be flat, and a reflection angle may be consistently maintained, and thus a propagation rate may be maintained uniformly upon a propagation movement of the electromagnetic waves, whereby it is possible to increase propagation efficiency of the electromagnetic waves.

A lower end of the robot arm 30 is rotatably disposed at base 1, and a base housing 3 is provided on the base 1, and the electromagnetic wave oscillator 50 separated from the beam irradiation head 10 is installed in the base housing 3 so as to be connected with the waveguide 40.

The reason why the electromagnetic wave oscillator 50 is provided in the base housing 3 is to dispose the electromagnetic wave oscillator 50 such that the weight of the electromagnetic wave oscillator 50 does not have an effect on the robot arm 30 as well as the beam irradiation head 10, and thus to enable a more precise control.

Therefore, since the electromagnetic wave oscillator 50 having a given weight is separated from the beam irradiation head 10, the weight of the beam irradiation head 10 and thus the supporting load of the robot arm 30 may be reduced, and the rotary motion of the robot arm 30 may be controlled more precisely, whereby it is possible to irradiate radiation on the same virtual surface for the stereotactic radiotherapy having the same center.

Further, since this makes it possible to irradiate a consistent amount of radiation on the affected part, it is possible to increase reliability of the apparatus.

Meanwhile, to reduce a size of the radiotherapy apparatus according to embodiments of the present invention, the waveguide 40 built in the robot arm 30 is installed in an installation hole formed in each arm of the robot arm 30 having a diameter of 5 cm.

At this time, if the electromagnetic waves of the electromagnetic wave oscillator 50 having an X-band frequency of 8 to 12 GHz (9.3 GHz) are used, it is possible to reduce a size of the waveguide 40 and thus the weight of the robot arm 30, and also it is possible to maintain a sufficient propagation rate of the electromagnetic waves.

The flexible and rotary-coupling waveguide pipe according to embodiments of the present invention may be applied even to other apparatus for inspecting and testing various materials, such as a non-destructive inspection apparatus.

Further, in FIG. 1, a circulation 80 is provided at an upper side of the robot arm, and a tube is installed at the robot arm.

According to the multi-joint radiotherapy apparatus using the flexible and rotary-coupling waveguide pipe, the electromagnetic wave oscillator and the beam irradiation head are separated from each other, and the waveguide propagating electromagnetic waves generated from the electromagnetic wave oscillator to the linear accelerating means is built in the robot arm supporting the beam irradiation head, such that the weight of the beam irradiation head and the load applied to the multi-joint robot arm may be reduced, and also it is possible to enable the stereotactic radiotherapy having the same center through the accurate operation control and thus always obtain a consistent dose, even though the beam is irradiated at any positions.

According to embodiments of the present invention, the rotary-coupling waveguide pipe is provided to the rotary-coupling part of each joint of a robot arm so that the motion at each joint is smoothly performed, and the waveguide has the linear pipe structure or the flexible pipe structure, and thus it is possible to provide the waveguide fitting motion characteristics.

According to embodiments of the present invention, the wave passage of the waveguide has the rectangular shape so as to reduce the propagation loss of the radiofrequency waves.

It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

What is claimed is:

1. A radiotherapy apparatus comprising:
a beam irradiation head;
a linear accelerator provided at the beam irradiation head;
a robot arm connected with the beam irradiation head and comprising a plurality of joints;
a waveguide built in the robot arm and connected with the linear accelerator; and
an electromagnetic wave oscillator configured to generate electromagnetic waves for providing the electromagnetic waves to the linear accelerator, wherein the electromagnetic wave oscillator is disposed outside the robot arm such that the robot arm does not carry the weight of the electromagnetic wave oscillator while the electromagnetic waves generated from the electromagnetic wave oscillator propagate to the linear accelerator through the wave guide installed through the robot arm.

2. The apparatus according to claim 1, further comprising a rotary-coupling waveguide pipe provided at one of the plurality of joints of the robot arm such that the electromagnetic waves generated from the electromagnetic wave oscillator propagate to the linear accelerator through the rotary coupling waveguide pipe.

3. The apparatus according to claim 1, further comprising a rotary-coupling waveguide pipe which comprises a body disposed at one of the plurality of joints, and a rotary-coupling part which is rotatably coupled at one end of the body.

4. The apparatus according to claim 1, wherein the waveguide has a straight pipe structure or a flexible pipe structure.

5. The apparatus according to claim 1, wherein a wave passage of the waveguide has a rectangular sectional shape.

6. The apparatus according to claim 1, further comprising a base housing coupled to a lower end of the robot arm, and wherein the electromagnetic wave oscillator is built in the base housing.
